(19) United States
(12) Patent Application Publication
(10) Pub. No.: US 2021/0212910 A1
(43) Pub. Date: Jul. 15, 2021

(54) Malodor Suppression in Hemp Derived Cannabidiol Used in Cosmetic Compositions

(71) Applicants: Francisco Tausk, Rochester, NY (US); Katharine Bryce, London (GB)

(72) Inventors: Francisco Tausk, Rochester, NY (US); Katharine Bryce, London (GB)

(21) Appl. No.: 17/140,194

(22) Filed: Jan. 4, 2021

Related U.S. Application Data
(60) Provisional application No. 62/959,349, filed on Jan. 10, 2020.

(51) Int. Cl. A61K 8/34; A61K 8/9717; A61K 8/06

(52) U.S. Cl. CPC A61K 8/347; A61K 8/9789; A61K 8/06; A61K 8/9717

(57) ABSTRACT

A cosmetic composition is provided including a hemp sourced cannabidiol with malodor control combination of a marine microalgae extract and a wild indigo extract.

### RELATED APPLICATIONS

This application claims benefit of U.S. Provisional Application No. 62/959,349 filed Jan. 10, 2020.

### BACKGROUND OF THE INVENTION

### Field of the Invention

The invention concerns malodor suppression in hemp derived cannabidiol containing cosmetic compositions.

### The Related Art

Recently cannabis has received much scientific and commercial attention. Legal restrictions have been relaxed, particularly where the psychoactive ingredient tetrahydrocannabidiol [THC] has been separated from useful non-harmful components such as cannabidiol [CBD].

For decades, US federal law did not differentiate hemp from other cannabis plants such as ones producing marijuana. In 1937 under the Marijuana Tax Act and more formally in 1970 under the Controlled Substances Act, cannabis of any kind was banned. Then in 2019 the US Farm Act exempted hemp derived CBD sources from being a controlled substance. There was a proviso that the hemp not contain more than 0.3% THC.

The cosmetics industry has become interested in formulating the newly available chemical into skin products. Typical of the pursuit are reports in the following patent literature.

U.S. Pat. No. 10,441,552 (Anastassov et al) discloses the anti-bacterial properties of cannabinoids. U.S. Pat. No. 9,326,927 (Zuccolo et al) reveals use of cannabinoids to color hair and skin. U.S. Pat. No. 9,415,082 (Davis) discusses use of hemp seed oil to treat skin conditions. US Patent Application 2019/0307719 (Karelis et al) presents cannabis compositions for treating a variety of skin disorders. US Patent Application 2019/0275095 (Spencer et al) reveals topical salves and lotions incorporating cannabinoids. US Patent Application 2019/0216695 (Kennedy) discloses anti-aging and skin tone lightening compositions which include cannabinoids. US Patent Application 2019/0247299 (Cameron et al) describes methods and compositions for treating eczema. US Patent Application 2018/0049971 (Druilhet) discloses a multi-purpose moisturizer which may contain hemp seed as an ingredient.

Background non-patent literature includes Cosmetics 2017, 4, 46 “The Potential Use of Marine Microalgae and Cyanobacteria in Cosmetics and Thalassotherapy” by Lourdes Mourelle et al.

During the work to develop cosmetic compositions based on hemp derived cannabidiol, we encountered a malodor problem. The scent was a hemp-like unpleasant aroma. Accordingly, we disclose herein formulation components that suppress the hemp-like aroma. Specifically we report that microalgae extract in combination with wild indigo extract efficiently suppress the hemp malodor.

### SUMMARY OF THE INVENTION

A cosmetic composition is provided which includes:
a) cannabidiol derived from hemp;
b) a marine microalgae extract;
c) a wild indigo extract; and
d) a cosmetically acceptable carrier.

Also provided is a method for reducing malodor associated with cannabidiol

derived hemp which includes mixing a combination of
a) cannabidiol derived from hemp;
b) a marine microalgae extract;
c) a wild indigo extract; and
d) a cosmetically acceptable carrier.

### DETAILED DESCRIPTION OF THE INVENTION

Now we have found chemicals that suppress the hemp-like aroma of hemp derived cannabidiol. Specifically we report that microalgae extract in combination with wild indigo extract efficiently suppresses the hemp malodor.

The first of the odor suppressant chemicals is within the marine microalgae extract family. Of particular efficacy are the extracts of Rhodosorus Marinus microalgae. The INCI name of the extract is Water (and) Propanediol (and) Rhodosorus Marinus Extract. Commercially, the microalgae extract is available from Givaudan SA under the trademark Mariliance®. See the Prospector® report on Mariliance® internet

www.ulprospector.com/en/na/PersonalCare/Detail/829/724171/Mariliance.

The microalgae extract may be present in amounts (exclusive of water/propanediol) ranging from 0.000001 to 5%, occasionally from 0.0001 to 1%, and even from 0.01 to 0.3% by weight of the composition.

In conjunction with the marine microalgae extract, the co-malodor suppressant is an extract of wild indigo. Of particular efficacy is Tephrosia Purpurea Seed Extract. The INCI name is Water (and) Propanediol (and) Tephrosia Purpurea Seed Extract. The material is commercially available from Givaudan SA under the trademark Neurophroline®. See The Prospector® report on Neurophroline® internet location

www.ulprospector.com/en/na/PersonalCare/Detail/829/653439/Neurophroline

The wild indigo extract material may be present in amounts (exclusive of water/propanediol) ranging from 0.000001 to 5%, occasionally from 0.0001 to 1%, and even from 0.01 to 0.3% by weight of the composition.

A third component is a hemp derived cannabidiol. There are 113 identified cannabinoids in cannabis plants and account for up to 40% of the plant's extracts. These extracts are oil-phase soluble. The word “Cannabidiol” as defined herein refers to an extracted oil soluble broad spectrum hemp derived oil (containing in amount from 30 to 95%, preferably from 50 to 90% and more preferably from 60 to 90% of cannabidiol itself by weight of the oil) and minor amounts (from 0 to 25% by weight of the oil) cannabidiol derivatives. As used in this application, cannabidiol is obtained from industrial hemp extract with a low amount of THC or from cannabis extract using high-CBD cannabis cultivars.

Amounts of the cannabidiol many range from 0.000001 to 1%, sometimes from 0.00001 to 1%, occasionally from 0.0001 to 0.1%, and even from 0.001 to 0.01% by weight of the cosmetic composition.

Relative weight amounts of the marine microalgae to the wild indigo extracts (exclusive of water/propanediol) may range from 10:1 to 1:5, occasionally from 2:1 to 1:2, and especially from 1.2 to 1.0. The cannabidiol in relative weight amounts to a combined weight amount of the marine microalgae plus wild indigo extracts (exclusive of water/propanediol) may range from 1:10 to 10:1, occasionally from 1:2 to 2:1, and even from 1:1.5 to 1:1.

Cosmetic compositions of this invention also include a cosmetically acceptable carrier. Amounts of the carrier may range from 1 to 99.9%, preferably from 70 to 95%, optimally from 80 to 90% by weight of the composition. Among the useful carriers are water, emollients, fatty acids, fatty alcohols, humectants, thickeners and combinations thereof. The carrier may be aqueous, anhydrous or an emulsion. Preferably the compositions are aqueous, especially water and oil emulsions of the W/O or O/W or triplex W/O/W variety. Water when present may be in amounts ranging from 5 to 95%, preferably 20 to 70%, optimally from 35 to 60% by weight.

Water when present as carrier or otherwise may advantageously be incorporated into the compositions as a deionized, sterilized or pasteurized liquid or can be heat treated or irradiated after having been mixed with other components of the composition. These treatments insure elimination of pathogenic microbes.

Emollient materials may serve as cosmetically acceptable carriers. These may be in the form of silicone oils, synthetic or natural esters, hydrocarbons, fatty acids and fatty alcohols. Amounts of the emollients may range anywhere from 0.1 to 95%, preferably between 1 and 50% by weight of the cosmetic composition.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes with viscosities of from about 5×10⁻⁶ to 0.1 m²/s at 25° C. Among the preferred nonvolatile emollients useful in the present compositions are the polydimethyl siloxanes having viscosities from about 1×10⁻⁵ to about 4×10⁻⁴ m²/s at 25° C.

Another class of nonvolatile silicones are emulsifying and non-emulsifying silicone elastomers. Representative of this category is Dimethicone/Vinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester emollients are:

1) Alkyl esters of saturated fatty acids having 10 to 24 carbon atoms. Examples thereof include behenyl neopentanoate, isononyl isonanonoate, isopropyl myristate and octyl stearate.

2) Ether-esters such as fatty acid esters of ethoxylated saturated fatty alcohols.

3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.

4) Wax esters such as beeswax, spermaceti wax and tribehenin wax.

5) Sugar ester of fatty acids such as sucrose polybehenate and sucrose polycottonseedate.

Hydrocarbons which are suitable cosmetically acceptable carriers include petrolatum, mineral oil, C₁₁-C₁₃ isoparaffins, and especially isohexadecane, available commercially as Permethyl 101A from Presperse Inc.

Fatty acids having from 10 to 30 carbon atoms may also be suitable as cosmetically acceptable carriers. Illustrative of this category are pelargonic, lauric, myristic, palmitic, stearic, isostearic, oleic, hydroxystearic and behenic acids.

Fatty alcohols having from 10 to 30 carbon atoms are another useful category of cosmetically acceptable carrier. Illustrative of this category are stearyl alcohol, lauryl alcohol, myristyl alcohol and cetyl alcohol.

Humectants of the polyhydric alcohol-type can be employed as cosmetically acceptable carriers. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of humectant may range anywhere from 0.5 to 50%, preferably between 1 and 15% by weight of the composition.

Thickeners can be utilized as part of the cosmetically acceptable carrier of compositions according to the present invention. Typical thickeners include Carbomers such as crosslinked acrylates (e.g. Carbopol 982®), hydrophobically-modified acrylates (e.g. Carbopol 1382®); cellulosic derivatives; and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin, alginate and combinations of these gums. Inorganics may also be utilized as thickeners, particularly clays such as bentonites, montmorillonites, illites and hectorites; fumed silicas; and silicates such as magnesium aluminum silicate (Veegum®). Amounts of the thickener may range from 0.0001 to 10%, usually from 0.001 to 1%, optimally from 0.01 to 0.5% by weight of the composition.

Sunscreens may be formulated into the compositions of this invention. They may be organic or inorganic. Included can be UVA and/or UVB spectrum protective ranges. Organic sunscreens will have at least one chromophoric group absorbing within the ultraviolet ranging from 290 to 400 nm, Chromophoric organic sunscreens may be divided into the following categories (with specific examples) including: p-Aminobenzoic acid, its salts and its derivatives (ethyl, isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,6-disulfonic and of 2-naphthol-6,8-disulfonic acids); Dihydroxynaphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzone, Sulisobenzone, Dioxybenzone, Benzoresorcinol, 2,2,4,4′-Tetrahydroxybenzophenone, 2,2′-Dihydroxy-4,4′-dimethoxybenzophenone, Octabenzone; 4-lsopropyldibenzoylmethane; Butylmethoxydibenzoylmethane; Etocrylene; and 4-isopropyl-dibenzoylmethane).

Particularly important sunscreens are: 2-ethylhexyl p-methoxycinnamate (available as Parsol MCX®), 4,4′-t-butyl methoxydibenzoylmethane (known commonly as Avobenzone, available as Parsol 1789®), octylsalicylate (available as Dermablock OS®), tetraphthalylidene dicamphor sulfonic acid (available as Mexoryl SX®), benzophenone-3 (Oxybenzone) and mixtures thereof.

Inorganic sunscreens are usually microfine particles of titanium dioxide and of zinc dioxide. “Microfine” is defined herein as average particle size ranging from 10 to 200 nm, usually from 20 to 100 nm.

Amounts of sunscreen may range from 0.01 to 20%, usually from 0.5 to 15%, and often from 4 to 12% by weight of the cosmetic composition.

Surfactants suitable for use may be those which can form emulsions and/or association structures. Surfactants can be categorized as being of the anionic, nonionic, cationic, or amphoteric type. The term “surfactants” are defined herein to include materials otherwise called “emulsifiers”.

Examples of surfactants which may be used in the compositions described herein include salts of C8-C22 alkyl chain compounds. Representative are sodium tallowate, sodium cocoate, sodium alkyl sulfate (e.g., sodium lauryl sulfate and sodium myristyl sulfate), sodium N-acyl sarcosinates (e.g., sodium N-lauroyl sarcosinate and sodium N-myristoyl sarcosinate), sodium dodecylbenzenesulfonate, sodium hydrogenated coconut fatty acid monoglyceride sulfate, sodium lauryl sulfoacetate and N-acyl glutamates (e.g., N-palmitoyl glutamate), N-methylacyltaurin sodium salt, N-methylacylalanine sodium salt, sodium alpha-olefin sulfonate and sodium dioctylsulfosuccinate; N-alkylaminoglycerols (e.g., N-lauryl-diamino-ethylglycerol and N-myristyldiaminoethyl glycerol), N-alkyl-N-carboxymethylammonium betaine and sodium 2-alkyl-1-hydroxyethylimidazoline betaine; polyoxyethylenealkyl ether, polyoxyethylene alkylaryl ether, polyoxyethylene lanolin alcohol, polyoxyethylene glyceryl monoaliphatic acid ester, polyoxyethylene sorbitol aliphatic acid ester, polyoxyethylene aliphatic acid ester, higher aliphatic acid glycerol ester, sorbitan aliphatic acid ester, and polyoxyethylenesorbitan aliphatic acid esters such as polyoxyethylenesorbitan monooleate and polyoxyethylene sorbitan monolaurate.

Oil continuous (W/O) emulsions are created by dimethicone copolyols. A suitable example of such an emulsifier is PEG-8 Dimethicone which arises from polyethylene glycol derivative of Dimethicone containing an average of 8 moles ethylene oxide. Another useful oil continuous phase emulsifier is Gransurf® 2106, a self-emulsifying elastomer gel. The INCI name for Gransurf® 2106 is Dimethicone (and) PEG-10 Dimethicone Crosspolymer available from Grant Industries. Cloud Point and the Draves Wetting Test are useful recognized tools to evaluate clarity and stability of the emulsions.

The surfactants/emulsifiers can be used at levels from 0.001% to 20%, preferably from 0.1% to 15%, more preferably from 1% to 10% and most preferably from 2% to 8% by weight of the cosmetic composition.

Preservatives may be incorporated into the cosmetic compositions to protect against the growth of potentially harmful microorganisms. Suitable traditional preservatives are alkyl esters of para-hydroxybenzoic acid. Other preservatives which have more recently come into use include hydantoin derivatives, propionate salts, sorbate salts and a variety of quaternary ammonium compounds. Cosmetic chemists are familiar with appropriate preservatives and routinely choose them to satisfy the preservative challenge test and to provide product stability. Particularly preferred preservatives are methylchloroisothiazolinone and methylisothiazolinone combinations, methyl paraben, propyl paraben, imidazolidinyl urea, sodium dehydroacetate and benzyl alcohol. Preservatives may be employed in amounts ranging from 0.01% to 2% by weight of the cosmetic composition.

Desquamation agents may be present. Illustrative are the monocarboxylic acids. Monocarboxylic acids may be substituted or unsubstituted with a carbon chain length of up to 16. Particularly preferred carboxylic acids are the alpha-hydroxycarboxylic acids, beta-hydroxycarboxylic or polyhydroxycarboxylic acids, The term “acid” is meant to include not only the free acid but also salts and C₁-C₃₀ alkyl or aryl esters thereof and lactones generated from removal of water to form cyclic or linear lactone structures. Representative acids are glycolic, lactic, malic and tartaric acids. A representative salt that is particularly preferred is ammonium lactate. Salicylic acid is representative of the beta-hydroxycarboxylic acids. Amounts of these materials when present may range from 0.01 to 15% by weight of the cosmetic composition.

Preferred desquamation agents may be selected from the group consisting of glycolic acid, lactic acid, salicylic acid, retinoic acid, retinol and mixtures thereof, and including salt forms thereof.

Antiperspirant skin care cosmetic compositions for use herein may include well known antiperspirant metal salts of aluminum, zinc, zirconium and zirconium aluminum mixtures of sulfates, chlorides, chlorohydroxides, tetrachlorohydrex glycinates, alums, formates, lactates, benzyl sulfonates, succinates, phenol sulfonates and the like. Typical levels of antiperspirant metal salts range from 1% to 35%, preferably from 1.5% to 25% by weight of the cosmetic composition.

Colorants may either be dyes or pigments. A distinction is usually made between a pigment, which is insoluble in its vehicle (resulting in a suspension), and a dye, which either is itself a liquid or is soluble in its vehicle (resulting in a solution). A colorant can act as either a pigment or a dye depending on the vehicle involved. In some cases, a pigment can be manufactured from a dye by precipitating a soluble dye with a metallic salt. The resulting pigment is called a lake pigment.

Among the more common dyes are Alizarin, Azophloxin, Chrysoidin, Congo Red, Fuchsin acid, Gentian violet, Janus green, Methyl Red, Naphthol Green, Naphthol Yellow, Rose Bengal, Sudan II, Titan Yellow and combinations thereof. Amongst pigments, titanium dioxide and aluminum lakes (aluminum salts of dyes) are most common. Amounts of the colorant may, according to the type of cosmetic product (lipstick, foundation, hair dye, etc) range from 0.000001 to 10%, usually from 0.01 to 5% by weight of the cosmetic composition.

Cosmetic compositions intended to be skin lighteners normally will be formulated with a skin lightening compound. Illustrative substances are placental extract, lactic acid, niacinamide, arbutin, kojic acid, ferulic acid, hydroquinone, resorcinol and derivatives including 4-substituted resorcinols and combinations thereof. Amounts of these substances may range from 0.1 to 10%, preferably from 0.5 to 2% by weight of the composition.

Also included may be such materials as resveratrol, alpha-lipoic acid, ellagic acid, kinetin, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B, Ceramide 6 and Ceramide 7) as well as pseudoceramides are useful. Amounts of these materials may range from 0.000001 to 10%, preferably from 0.0001 to 1% by weight of the composition.

The cosmetic compositions may contain a peptide active selected from pentapeptides, derivatives of pentapeptides, and mixtures thereof. As used herein, “pentapeptides” refers to both the naturally occurring pentapeptides and synthesized pentapeptides. A pentapeptide derivative-containing composition is Matrixyl®, which is commercially available from Sederma, France. The pentapeptides and/or pentapeptide derivatives are preferably included in amounts of from 0.000001% to 20%.

Cosmetic compositions may include vitamins. Illustrative vitamins are Vitamin A (retinol), Vitamin B₂, Vitamin B₃ (niacinamide), Vitamin B₆, Vitamin B12, Vitamin C, Vitamin D, Vitamin E, Vitamin K and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. A particularly suitable Vitamin B₆ derivative is Pyridoxine Palmitate. Flavonoids may also be useful, particularly glucosyl hesperidin, rutin, and soy isoflavones (including genistein, daidzein, equal, and their glucosyl derivatives) and mixtures thereof. Total amount of vitamins or flavonoids when present may range from 0.0001 to 10% by weight of the composition.

The cosmetic compositions may be formulated into a wide variety of product types that include but are not limited to solutions, suspensions, lotions, creams, gels, toners, sticks, sprays, ointments, cleansing liquid washes and solid bars, shampoos and hair conditioners, pastes, foams, powders, mousses, shaving creams, wipes, strips, patches (transdermal or non-transdermal), electrically-powered patches, wound dressing and adhesive bandages, hydrogels, film-forming products, facial and skin masks, make-up such as foundations, eye liners, and eye shadows, and the like.

Except in the examples, or where otherwise explicitly indicated, all numbers in this description indicating amounts of material or conditions of reaction, physical properties of materials and/or use are to be understood as modified by the word “about.” All amounts are by weight of the final cosmetic composition, unless otherwise specified.

It should be noted that in specifying any range of concentration or amount, any particular upper concentration can be associated with any particular lower concentration or amount.

In many of the Examples which follow, ingredients are listed without specific concentrations. These listings are to be considered as progressing from highest amount (e.g. water) to lowest ending at the fragrance. Levels of ingredients below fragrance may be in random concentration order.

For the avoidance of doubt, the word “comprising” is intended to mean “including” but not necessarily “consisting of” or “composed of.” In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

“Cosmetic Composition” as used herein, is meant to include a composition for topical application to skin or hair of mammals, especially humans. Such a composition may be generally classified as leave-on or rinse off, and includes any product applied to a human body for improving appearance, cleansing, odor control or general aesthetics.

### Example 1

A clarifying facial mask of the present invention is formed with the following ingredients (INCI Nomenclature): Water, Kaolin, Glyceryl Stearate SE, Glycerin, Propanediol, Heptyl Undecylenate, Charcoal Powder, Caprylic/Capric Triglyceride, Magnesium Aluminum Silicate, Cetyl Alcohol, Sodium Benzoate, Sodium Stearoyl Glutamate, Potassium Sorbate, Xanthan Gum, Lactic Acid, Pentylene Glycol, Rhodosorus Marinus Extract, Citric Acid, Tephrosia Purpurae Seed Extract, Glycolic Acid, Titanium Dioxide, Quartz, Barosma Betulina Leaf Extract, Mandelic Acid, Curcubita Pepo (Pumpkin) Fruit Extract, Ganoderma lucidum (Mushroom) Extract, Benzoic Acid, and Cannabidiol.

### Example 2

A nutrient facial mask of the present invention is formed with the following ingredients (INCI Nomenclature): Water, Kaolin, Glycerin, Cetearyl Alcohol, Prunus Amygdalus Dulcis Oil, Stearic Acid, Glyceryl Stearate SE, Butyrospermum parkii (Shea) Butter, Magnesium Aluminum Silicate, Montmorillonite, Illite, Benzyl Alcohol, Xanthan Gum, Glyceryl Caprylate, Cannabidiol, Propanediol, Fragrance, Hydrolyzed Sodium Hyaluronate, Benzoic Acid, Melia Azadirachta Flower Extract, Melia Azadirachta Leaf Extract, Dehydroacetic Acid, Glyceryl Undecylenate, Titanium Dioxide, Rhodosorus Marinus Extract, Camellia Olifiera Leaf Extract, Curcuma longa (Tumeric) Root Extract, Quartz, Tephrosia Purpurea Seed Extract, Tocopherol and Citric Acid.

### Example 3

A gel moisturizer of the present invention is formed with the following ingedients (INCI Nomenclature): Water, Diheptyl Succinate, Glycerin, Ammonium Acryloyldimethyltaurate/VP Copolymer, Avena sativa (Oat) Kernel Flour, Hydroxyethyl Acrylate/Sodium Acryloyldimethyl Taurate Copolymer, Niacinamide, Propanediol, Sodium Benzoate, Salicylic Acid, Citric Acid, Potassium Sorbate, Pentylene Glycol, Rhodosorus Marinus Extract, Tephrosia Purpurea Seed Extract, Glycolic Acid, Capryloyl Glycerin/Sebacic Acid Copolymer, Bambusa Vulgaris Extract, Curcuma longa (Turmeric) Root Extract, Benzoic Acid, Sodium Hyaluronate, and Cannabidiol.

### Example 4

A stay balanced gel moisturizer of the present invention is formed with the following ingredients (INCI Nomenclature): Water, Diheptyl Succinate, Glycerin, Ammonium Acryloyldimethyltaurate/VP Copolymer, Propanediol, Niacinamide, Hydroxyethyl Acrylate/Sodium Acryloyldimethyl Taurate Copolymer, Avena sativa (Oat) Kernel Flower, Sodium Benzoate, Fragrance, Potassium Sorbate, Cannabidiol, Salicylic Acid, Citric Acid, Rhodosorus Marinus Extract, Tephrosia Purpurea Seed Extract, Glycolic Acid, Capryloyl Glycerin/Sebacic Acid Copolymer, Sorbitan Isostearate, Cannabis Sativa Seed Oil, Bambusa Vulgaris Extract, and Sodium Hyaluronate.

### Example 5

A skin serum of the present invention is formed with the following ingredients (INCI Nomenclature): Water, 3-O-Ethyl Ascorbic Acid, Glycerin, Ethoxydiglycol, Propanediol, Tamarindus Indica Seed Polysaccharide, Sodium Benzoate, Carrageenan, Tocopherol, Ferulic Acid, Potassium Sorbate, Glucose, Tephrosia Purpurea Seed Extract, Pentylene Glycol, Rhodosorus Marinus Extract, Glycyrrhiza glabra (Licorice) Root Extract, Benzoic Acid, and Cannabidiol.

### Example 6

A repair and rewind power skin serum of the present invention is formed with the following ingredients (INCI Nomenclature): Water, Glycerin, Simmondsia chinensis (Jojoba) Seed Oil, Propanediol, Cannabidiol, Xanthan Gum, Sodium Benzoate, Vaccinium Myrtillus Fruit Extract, Fragrance, Potassium Sorbate, Cannabidiol, Saccharum officinarum (Sugar Cane) Extract, Rhodosorus Marinus Extract, Cannabis Sativa Seed Oil, Tephrosia Purpurea Seed Extract, Sorbitol, Citrus aurantium dulcis (Orange) Fruit Extract, Citrus limon (Lemon) Fruit Extract, Hippophae Rhamnoides Fruit Oil, Ceratonia Silqua Gum, Acer Saccharum Sugar Maple) Extract, Glycyrrhiza glabra (Licorice) Root Extract, Palmitoyl Tripeptide-5, 1,2-Hexanediol, Caprylyl Glycol, Gold, Ascorbic Acid, and Glutathione.

### Example 7

A skin elixir oil of the present invention is formed with the following ingredients (INCI Nomenclature): Helianthus annuus (Sunflower) Seed Oil, Cannabis Sativa Seed Oil, Caryodendron Orinocense Seed Oil, Simmondsia chinesis (Jojoba) Seed Oil, Caprylic/Capric Triglyceride, Oryza sativa (Rice) Bran Extract, Linoleic Acid, Helianthus annuus (Sunflower) Extract, Rosmarinus Officinalis, (Rosemary) Leaf Extract, Salicornia Herbacea Extract, Tocopherol, Linolenic Acid, Ganoderma lucidum (Mushroom) Extract, Panax Ginseng Root Extract, Withania Somnifara Root Extract, Tephrosia Purpurea Seed Extract, and Rhodosorus Marinus Extract.

### Example 8

A nourish and soothe skin elixir oil of the present invention is formed with the following ingredients (INCI Nomenclature): Helianthus annuus (Sunflower) Seed Oil, Prunus armeniaca (Apricot) Kernel Oil, Oleic/Linoleic/Linolenic Polyglycerides, Caryodendron Orinocense Seed Oil, Oryza sativa (Rice) Bran Extract, Cannabidiol, Glycerin, Linoleic Acid Water, Fragrance, Caprylic/Capric Triglyceride, Helianthus (Sunflower) Extract, Propanediol, Cannabis Sativa Seed Oil, Rosmarinus officinalis (Rosemary) Leaf Extract, Tocopherol, Rhodosorus Marinus Extract, Tephrosia Purpurea Seed Extract, Salicomia Herbacea Extract, Linolenic Acid, Ganoderma lucidum (Mushroom) Extract, Panax Ginseng Extract, and Withania Somnifera Root Extract.

### Example 9

A cleansing balm of the present invention is formed with the following ingredients (INCI Nomenclature): Helianthus annuus (Sunflower) Seed Oil, Glycerin, Caprylic/Capric Triglyceride, Water, Sucrose Laurate, Borago officinalis (Borage) Seed Oil, Sodium Benzoate, Ricinus communis (Castor) Seed Oil, Propanediol, Sodium Hyaluronate, Potassium Sorbate, Hippophae Rhamnoides Fruit Oil, Moringa Oleifera Seed Oil, Hydrogenated Castor Oil, Pentylene Glycol, Rhodosorus Marinus Extract, Tephrosia Purpurea Seed Extract, Benzoic Acid, and Cannabidiol.

### Example 10

A skin contour lifting cream of the present invention is formed with the following ingredients (INCI Nomenclature): Water, Cetyl Ethylhexanoate, Glyceryl Stearate SE, isopropyl Myristate, Stearic Acid, Cetyl Alcohol, Benzyl Alcohol, Hydrolyzed Collagen, Propanediol, Glycerin, Bakuchiol, Sodium Stearoyl Glutamate, Carbomer, Sorbitan Stearate, Benzoic Acid, Disteardimonium Hectorite, Sodium Hydroxide, Plukenetia Volubilis Seed Oil, Dehydroacetic Acid, Tephrosia Purpurea Seed Extract, Sodium Phytate, Pentylene Glycol, Rhodosorus Marinus Extract, Ceramide Ng, Sodium Benzoate, Bacillus Ferment, Hydroxypropyl Cyclodextrin, Potassium Sorbate, Tocopherol, Palmitoyl Tripeptide-38, and Cannabidiol.

### Example 11

A redefining skin contour lifting cream of the present invention is formed with the following ingredients (INCI Nomenclature): Water, Glyceryl Stearate SE, Isopropyl Myristate, Stearic Acid, Glyceryl Behenate, Heptyl Undecylenate, Cetyl Alcohol, Capryloyl Glycerin/Sebacic Acid Copolymer, Diheptyl Succinate, Propanediol, Caprylic/Capric Triglyceride, Glyceryl Caprylate, Sodium Stearoyl Glutamate, Glycerin, Carbomer, Cannabidiol, Bakuchiol, Fragrance, Caprylhydroxamic Acid, Rhodosoros Marinus Extract, Plukenetia Volubilis Seed Oil, Tephrosia Purpurea Seed Extract, Sodium Phytate, Sodium Hydroxide, Cannabis Sativa Seed Oil, Collagen Amino Acids, Melia Azadirachte Flower Extract, Leuconostoc/Radish Root Ferment Filtrate, Melia Azadirachte Leaf Extract, Coccinia Indica Fruit Extract, Curcuma longa (Tumeric) Root Extract, Hydroxypropyl Cyclodextrin, Ocimum basilicum (Basil) Flower/Leaf Extract, Ocimum Sanctum Leaf Extract, Solanum melongeria (Eggplant) Fruit Extract, Aloe Barbadensis Flower Extract, Palmitoyl Tripeptide-38, and Cannabidiol.

### Example 12

A daily facial wash of the present invention is formed with the following ingredients (INCI Nomenclature): Water, Cetearyl Alcohol, Cocoglycerides, C12-15 Alkyl Benzoate, Propanediol, Glycerin, Cetyl Palmitate, Sorbitan Olivate, Sorbitan Palmitate, Jojoba Esters, Simmondsia chinesis (Jojoba) Seed Oil, Cetearyl Glucoside, Helianthus annuus (Sunflower) Seed Wax, Benzyl Alcohol, Xanthan Gum, Panthenol, Tocopherol, Benzoic Acid, Acacia Decurrens Flower Wax, Pentylene Glycol, Rhodosorus Marinus Extract, Tephrosia Purpurea Seed Extract, Dehydroacetic Acid, Butylene Glycol, Polyglycerin-3, Citrus aurantium bergamia (Bergamot) Fruit Oil, Eucalyptus Radiata Leaf/Stem Oil, Matricaria recutita (Chamomile) Extract, Rosmarinus officianlis (Rosemary) Leaf Oil, Vitis vinifera (Grape) Seed Extract, Sodium Benzoate, Citric Acid, and Cannabidiol.

### Example 13

A re-energizing daily minifacial wash of the present invention is formed with the following ingredients (INCI Nomenclature): Water, C13-C15 Alkane, Cetearyl Alcohol, Helianthus annuus (Sunflower) Seed Oil, Glycerin, Propanediol,Cetyl Palmitate, Caprylic/Capric Triglyceride, Cetearyl Glucoside, Sorbitan Olivate, Sodium Palmitate, Jojoba Esters, Benzyl Alcohol, Helianthus annuus (Sunflower) Seed Wax, Benzyl Alcohol, Lactobacillus/Pumpkin Ferment Extract, Xanthan Gum, Cannabinol, Fragrance, Benzoic Acid, Tocopheryl Acetate, Acacia Decurrens Flower Wax, Dehydroacetic Acid, Panthenol, Polyglycerin-3, Sodium Hydroxide, Cannabis Sativa Seed Oil, Matricaria recutita (Chamomile) Extract, Rhodosorus Marinus Extract, Tephrosia Purpurea Seed Extract, Vitis vinifera (Grape) Seed Extract, Tocopherol, and Citric Acid.

### Example 14

An eye cream of the present invention is formed with the following ingredients (INCI Nomenclature): Water, Helianthus annuus (Sunflower) Seed Oil, Octyldodecanol, Glycerin, Synthetic Beeswax, Glyceryl Stearate, Polymethylsilsesquioxane, Stearath-2, Cetyl Alcohol, Stearyl Alcohol, Ethylhexylglycerin, Propanediol, Carbomer, Caffeine, Niacinamide, Sodium Benzoate, Xanthan Gum, Pentylene Glycol, Potassium Sorbate, Pentylene Glycol, Rhodosorus Marinus Extract, Tephrosia Purpurea Seed Extract, Magnesium Aspartate, Zinc Gluconate, Arnica Montana Extract, Glycyrrhiza glabra (Licorics) Root Extract, Phospholipids, Copper Gluconate, Sphingolipids, Benzoic Acid, Lactic Acid, Polysorbate-20, Palmitoyl Tripeptide-1, Palmitoyl Tetrapeptide-7, and Cannabidiol.

### Example 15

A nutrient skin mask of the present invention is formed with the following ingredients (INCI Nomenclature): Water, Kaolin, Microcrystalline Wax, Monmorillonite, Glyceryl Stearate, Illite, Helianthus annuus (Sunflower) Seed Oil, Butyrospermum parkii (Shea) Butter, Iron Oxides, Stearic Acid, Magnesium Aluminum Silicate, Cetyl Alcohol, Bismuth Oxychlorides, Mica, Sodium Ascorbyl Phosphate, Bisabolol, Sodium Benzoate, Chromium Oxide Greens, Propanediol, Xanthan Gum, Potassium Sorbate, Allantoin, Hydrolyzed Sodium Hyaluronate, Calcium Stearate, Hydrogenated Vegetable Oil, Prunus amygdalus dulcis (Sweet Almond) Oil, Pentylene Glycol, Rhodosorus Marinus Extract, Tephrosia Purpurea Seed Extract, Titanium Dioxide, Hydrogenated Lecithin, Farnesol, Ganoderma lucidum (Mushroom) Extract, Quartz, Pichia/Resveratrol Ferment Extract, Camellia Sinensis Leaf Powder, Benzoic Acid, Tocopherol, Citric Acid, and Cannabidiol.

### Example 16

An enzymatic cleanser of the present invention is formed with the following ingredients (INCI Nomenclature): Water, Sodium Lauroyl Methyl Isethionate, Glycerin, Stearic Acid, Sodium Cocoyl Isethionate, Glyceryl Stearate, Glycol Distearate, Cetyl Alcohol, Stearyl Alcohol, Propanediol, Coconut Acid, Guar Hydroxypropyltrimonium Chloride, Sodium Methyl Isethionate, Lauric Acid, Sodium Isethionate, Sodium Benzoate, Potassium Sorbate, Xanthan Gum, Pentylene Glycol, Rhodosorus Marinus Extract, Sodium Laurate, Tephrosia Purpurea Seed Extract, Citric Acid, Tocopheryl Acetate, Sodium Phytate, Papain, Glycolic Acid, Pentylene Glycol, Benzoic Acid, Salix alba (Willow) Bark Extract and Cannabidiol.

### Example 17

Another illustration of an enzymatic cleanser of the present invention is formed with the following ingredients (INCI Nomenclature): Water, Cetyl Alcohol, Cocamidopropyl Hydroxy Sultaine, Glycerin, Glycol Distearate, Stearic Acid, Sodium Chloride, Propanediol, Menthoxypropanediol, Sodium Benzoate, Xanthan Gum, Guar Hydroxypropyltrimonium Chloride, Potassium Sorbate, Cocamidopropyl Dimethylamine, Fragrance, Citric Acid, Sodium Phytate, Cannabidiol, Rhodosorus Marinus Extract, Tocopherol Acetate, Lactobacillus/Pumpkin Ferment Extract, Mandelic Acid, Lactobacillus/Papaya Fruit Ferment Extract, Glycolic Acid, Cannabis Sative Seed Oil,Tephrosia Purpurea Seed Extract, Citrullus lanatus (Watermelon) Fruit Extract, Citrus grandis (Grapefruit) Seed Extract, Salix alba (Willow) Bark Extract, and Leuconostoc/Radish Root Ferment Filtrate.

### Example 18

A facial mist of the present invention is formed with the following ingredients (INCI Nomenclature): Water, Propylene Glycol, Propanediol, Sodium Benzoate, 3-O-Ethyl Ascorbic Acid, Potassium Sorbate, Sodium Hyaluronate, Pentylene Glycol, Rhodosorus Marinus Extract, Tephrosia Purpurea Seed Extract, Glycerin, Cucumis sativus (Cucumber) Extract, Benzoic Acid, and Cannabidiol.

### Example 19

An illustrative skin care product for treating stressed skin has the formula below.

| Component | Weight % |
|---|---|
| Cannabidiol | 0.10 |
| Mariliancel ® microalgae extract | 0.30 |
| Neurophroline ® tephrosia purpurea | 0.20 |
| Niacinamide | 4.00 |
| Isohexadecane | 3.00 |
| Isopropyl isostearate | 2.00 |
| Sucrose Polycottonseedate | 0.70 |
| Polymethylsilsesquioxane | 0.25 |
| Cetearyl Glucoside/Cetearyl Alcohol | 0.25 |
| Behenyl Alcohol | 0.40 |
| Ethyl Paraben | 0.20 |
| Propyl Paraben | 0.10 |
| Cetyl Alcohol | 0.30 |
| Stearyl Alcohol | 0.45 |
| PEG-100 Stearate | 0.10 |
| Glycerin | 8.50 |
| Titanium Dioxide | 0.60 |
| Polymethacrylate | 2.00 |
| Dimethicone/Dimethiconol | 2.00 |
| Water | to 100 |

The water phase components are combined in a suitable vessel and heated to 75° C. In a separate suitable vessel, combine the oil phase ingredients and heat to 75° C. Next add the oil phase to the water phase and mill the resulting emulsion (e.g., with a Tekmar™ T-25 mill). Then, add the thickener to the emulsion and cool the emulsion to 45° C. while stirring. At 45° C., add the remaining components. Cool the product and stir to 30° C. and pour into suitable containers.

### Example 20

A still further illustration of skin treatment compositions is outlined in the Table below.

| Component | Weight % |
|---|---|
| Water | Balance to 100% |
| Glycerin | 30 |
| Denatured Alcohol | 10 |
| Dimethicone | 8 |
| Isononyl Isononanoate | 8 |
| Ascorbyl Glucoside | 3 |
| Biosaccharide Gum-1 | 3 |
| Caprylyl Glycol | 3 |
| Carbomer | 2 |
| Colorant | 1 |
| Dimethiconol | 1 |
| Menthoxypropanediol | 1 |
| Panthenol | 0.8 |
| Sodium Hydroxide | 0.8 |
| Triethanolamine | 0.6 |
| Xanthan Gum | 0.5 |
| Tetrasodium EDTA | 0.5 |
| Fragrance | 0.5 |
| Mariliance ® Rhodosorus Marinus Extract | 0.5 |
| Neurophroline ® Tephrosia Purpurea Extract | 0.3 |
| Cannabidiol | 0.1 |

### Example 21

Still another illustration of skin treatment compositions is outlined in the Table below.

| Component | Weight % |
|---|---|
| Water | Balance to 100% |
| Glycerin | 20 |
| Niacinamide | 10 |
| Isohexadecane | 8 |
| Dimethicone | 7.5 |
| Isopropyl Isostearate | 4 |
| Stearyl Alcohol | 4 |
| Polyacrylamide | 3 |
| Panthenol | 2 |
| Tocopheryl Acetate | 1 |
| Retinyl Propionate | 1 |
| Mariliance ® Rhodosorus Marinus Extract | 1 |
| Neuropholine ® Tephrosia Purpurea Seed | 0.8 |
| Cannabidiol | 0.8 |
| Triethanolamine | 0.8 |
| Tapioca Starch | 0.5 |
| Tetrasodium EDTA | 0.5 |
| Fragrance | 0.5 |
| Dimethiconol | 0.4 |
| Cetearyl Alcohol | 0.4 |
| Titanium Dioxide | 0.25 |
| Disodium Malonate | 0.22 |
| Potassium Sorbate | 0.20 |

While the present compositions and methods have been described with reference to the specific variations thereof, it should be understood by those skilled in the art that various changes may be made and equivalents may be substituted without departing from the true spirit and scope of the compositions and methods described herein. In addition, any modifications may be made to adapt a particular situation, material, composition of matter, process, process step or steps, to the objective, spirit and scope of the compounds and methods described herein. All patents and publications cited above are hereby incorporated by reference.

What is claimed is:

1. A cosmetic composition comprising:
a) cannabidiol derived from hemp;
b) a marine microalgae extract;
c) a wild indigo extract; and
d) a cosmetically acceptable carrier.

2. The cosmetic composition according to claim 1 wherein the microalgae extract is Rhodosorus Marinus extract.

3. The cosmetic composition according to claim 1 wherein the wild indigo extract is Tephrosia Purpurea Seed extract.

4. The cosmetic composition according to claim 1 wherein the cosmetically acceptable carrier is selected from the group consisting of water, emollients, humectants, thickeners and combinations thereof.

5. The cosmetic composition according to claim 1 wherein the carrier is a water and oil emulsion.

6. The cosmetic composition according to claim 1 wherein the marine microalgae extract is present in amounts ranging from 0.000001 to 5% by weight of the composition.

7. The cosmetic composition according to claim 6 wherein the wild indigo extract is present in amounts ranging from 0.000001 to 5% by weight of the composition.

8. The cosmetic composition according to claim 7 wherein the cannabidiol is present in amounts ranging from 0.000001 to 1% by weight of the composition.

9. The cosmetic composition according to claim 1 further comprising from 1 to 99.9% by weight of the composition of water in a form selected from the group consisting of deionized water, sterilized water and pasteurized water.

10. A method for reducing malodor associated with cannabidiol derived hemp, the method comprising the step of mixing a combination of
a) cannabidiol derived from hemp;
b) a marine microalgae extract;
c) a wild indigo extract; and
d) a cosmetically acceptable carrier.
